(19) United States
(12) Patent Application Publication
(10) Pub. No.: US 2009/0209980 A1
(43) Pub. Date: Aug. 20, 2009

(54) SUTURING AND KNOT-TYING DEVICE

(75) Inventor: Jerry Harris, Shreveport, LA (US)

Correspondence Address: WOMBLE CARLYLE SANDRIDGE & RICE, PLLC, ATTN: PATENT DOCKETING, P.O. BOX 7037, ATLANTA, GA, 30357-0037, US

(73) Assignee: Harris Research, LLC., Shreveport, LA (US)

(21) Appl. No.: 12/372,182

(22) Filed: Feb. 17, 2009

Related U.S. Application Data
(60) Provisional application No. 61/029,760, filed on Feb. 19, 2008.

(51) Int. Cl. A61B 17/062

(52) U.S. Cl. 606/144

(57) ABSTRACT

A tool for use in placing sutures and tying knots is disclosed. The tool has a handle for grasping by a user of the tool, an elongate neck extending from the handle, and a moveable head attached to the neck. A needle is operatively connected to the head. The needle is moveable between a retracted position and an extended position. A needle drive mechanism is provided for moving the needle relative to the head, while a head positioning mechanism is provided for moving the head relative to the handle.

### CROSS-REFERENCE TO RELATED APPLICATIONS

This application claims the benefit of U.S. provisional Application No. 61/029,760, filed on Feb. 19, 2008, the entire disclosure of which is incorporated herein by reference.

### BACKGROUND OF THE DISCLOSURE

The present disclosure generally relates to medical tools, and more particularly to a surgical tool for placing sutures in a patient and tying knots.

In performing surgical procedures, surgeons often rely on a existing methods of manually suturing and tying knots that are slow, time consuming and tedious. Such existing methods include the use of tools such as small curved needles, needle drivers, tweezers, and clamps to secure sutures and close external wounds. In conventional methods, a patient's wounds can be left open longer than desired, which increases the risk of infection or other complications. Also, existing methods further can cause fatigue and repetitive physical strain ailments or injuries for surgeons when manually manipulating existing tools to suture wounds and tie knots.

It therefore can be seen that a need exists for surgical tools that addresses the foregoing and other related and unrelated problems in the art.

### SUMMARY OF THE DISCLOSURE

Briefly described, in one aspect, the disclosure generally is directed to a tool for use in placing sutures for closing a wound and tying knots such as during surgical operations. The tool comprises a handle, an elongate neck extending from the handle, and a moveable head attached to the neck. A needle can be operatively connected to the head and typically is moveable between a retracted position and an extended position. A needle drive mechanism is provided for holding and moving the needle relative to the head, and a head positioning system further is provided for moving the head relative to the handle.

In another aspect, the disclosure is generally directed to a method of assisting in applying sutures or tying knots during surgical operations as shown or described herein.

According to common practice, the various features of the drawings discussed below are not necessarily drawn to scale. Dimensions of various features and elements in the drawings may be expanded or reduced to more clearly illustrate the embodiments of the disclosure.

### BRIEF DESCRIPTION OF THE DRAWINGS

FIG. 1 is a perspective view of a suturing and knot-tying device, according to one embodiment of the disclosure.

FIG. 2 is a side view illustrating the neck and wires of the head positioning system of the device.

FIG. 3A is a cut-away view of the handle of the device.

FIG. 3B is a cut-away view of the handle of the device according to an alternate embodiment, with parts broken away.

FIG. 4 is a perspective view of the neck of the device.

FIG. 5 is a perspective view, with parts broken away, of the head of the device.

FIG. 6 is a perspective view of a needle drive mechanism of the device.

FIG. 7 shows a needle groove plate of the device.

FIG. 8 shows a cover of the head.

FIG. 9 is a partial side view showing the neck and the head of the device.

FIG. 10 is a side elevational view of the device.

FIGS. 11-13 are side views illustrating the pivoting of the head of the device.

Corresponding parts are designated by corresponding reference numbers throughout the drawings.

### DETAILED DESCRIPTION OF THE EXEMPLARY EMBODIMENTS

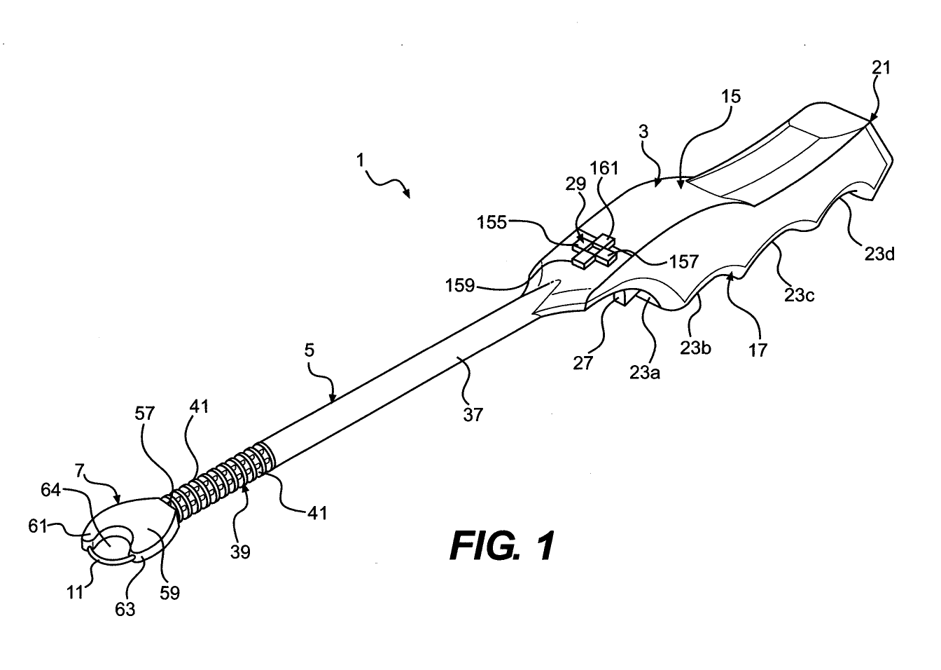

FIGS. 1-10 illustrate a suturing and knot-tying device or tool 1 according to exemplary embodiments of the present invention. In use, the tool 1 can be used for placing sutures or tying knots in a surgical environment or other non-surgical environments, utilizing attachment via surgical threads or other materials. As illustrated in FIG. 1, in one embodiment, the tool 1 generally includes a handle 3 for grasping by a user, an elongated neck portion 5 extending distally from the handle, and a moveable head section 7 attached to the free end of the neck. In the illustrated embodiment of FIGS. 1 and 5-9, the head 7 generally can house a needle 11, here shown as having a substantially arcuate configuration or shape, and which can be attached to surgical thread (not shown) or other material. The needle 11 is manipulated by the operation of the tool 1 as needed to create sutures and/or to tie knots within the suturing thread or other material. The head 7 further generally is capable of rotational movement as well as pivotal movement upon selective actuation by a user of the tool 1 to position the needle in a desired position as needed for performing a suturing or sewing operation with the tool.

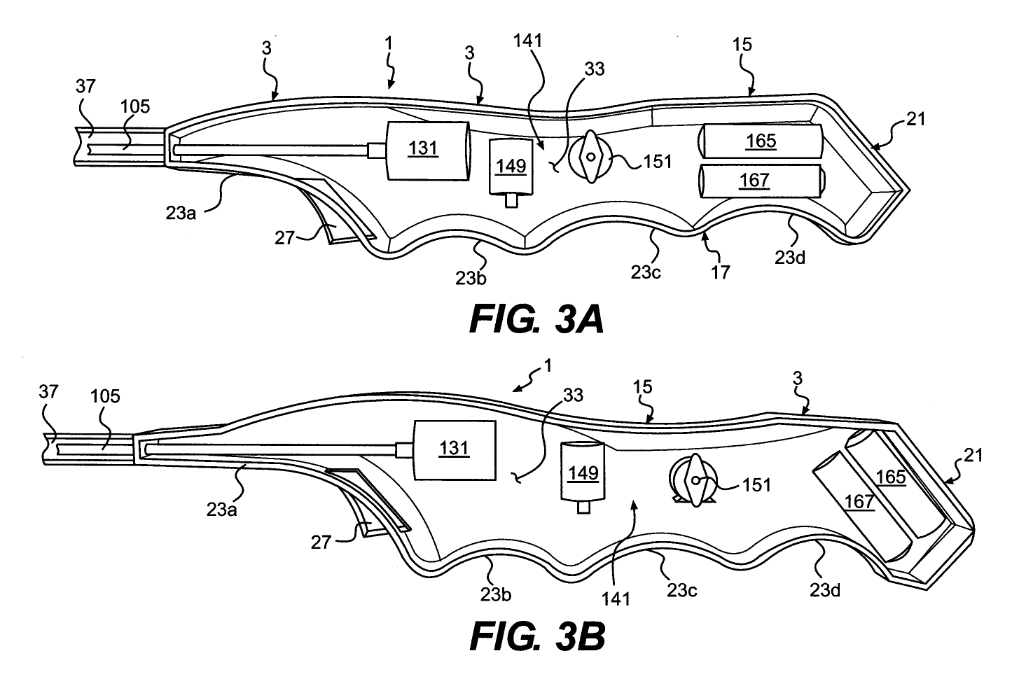

As illustrated in FIGS. 1 and 3A-3B, the handle 3 of the tool 1 generally is ergonomically shaped to be easily grasped and controlled by a user. The handle 3 will have a top surface 15, a bottom surface 17, and a rear portion 21 that can be shaped to fit comfortably into the palm of a user's hand when the handle is grasped. The bottom surface 17 also can include a series of grooves 23a-d (four grooves are shown here, although more or fewer grooves also can be provided) shaped for receiving the fingers of a user's hand when the rear portion 21 is grasped in the palm of the user's hand. The forward groove 23a can include a control mechanism 27 (e.g., a switch or trigger) for activation by a finger (e.g., index finger) of the user. As shown in FIG. 1, the top surface 15 also can include a separate control mechanism 29 (e.g., four-way/4-axis push button controller) that can be located along the top surface of the handle in a position for activation by a finger (e.g., thumb) of the user when the handle 3 is grasped. In one embodiment, the handle 3 generally is hollow, with a space 33 for housing various control mechanisms of the tool 1 as illustrated in FIGS. 3A-3B. The handle 3 may be made of injection-molded material (e.g., plastic) and can be formed by conventional injection-molding operations, or the handle can include other materials formed by other processes. The handle 3 further typically can be configured for use by either a left-handed user or a right-handed user without altering the configuration of the tool 1.

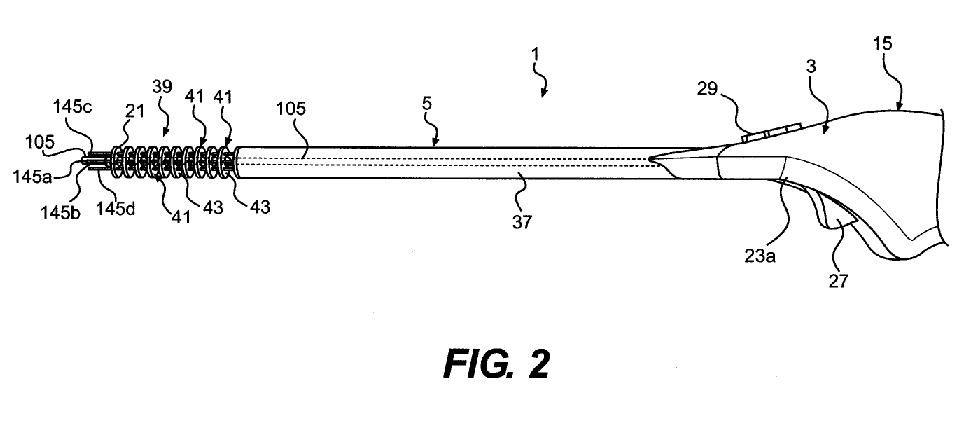

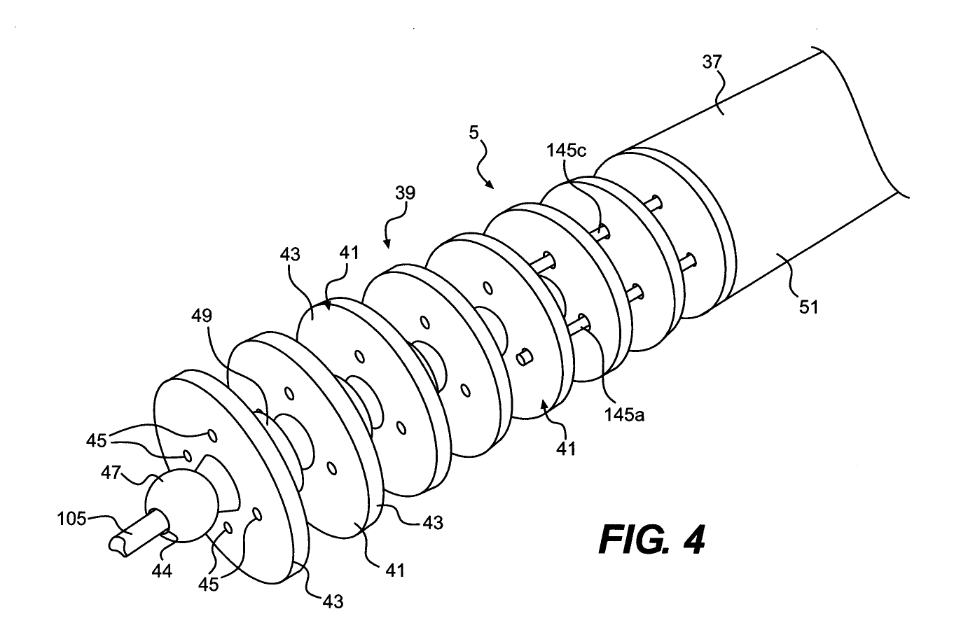

As further illustrated in FIGS. 1-2, the neck 5 of the tool generally will be received within and/or otherwise attached to a forward or front end of the handle and will extend or project distally therefrom. In one embodiment, the neck 5 includes a rigid portion 37 adjacent the handle 3 and flexible portion 39 adjacent the head 7. In the illustrated embodiment, the flexible portion 39 of the neck is an articulated section that includes a series of segments 41 operatively connected by a ball and plate connector configuration to allow movement of the head 7 relative to the handle 3. As shown in FIG. 4, each segment 41 includes a disk 43 generally having a series of openings 45, a male connecting portion 47 (e.g., ball) on one side of the disk, and a female connecting portion 49 (e.g., plate) on the other side of the disk. The segments 41 are movably connected by inserting the male connecting portion 47 of a segment into the corresponding female connecting portion 49 of an adjacent segment. In the illustrated embodiment, the neck 5 is shown in FIGS. 1, 2 and 4 with approximately eight-eleven segments 41, although it will be understood by those skilled in the art that the flexible portion of the neck could include more or less segments 41 than are presently illustrated. Further, the segments 41 can include other articulatable, flexible connections between the segments that is other than the ball and plate configuration of the illustrated embodiment.

The rigid portion 37 of the neck 5 further is shown in FIGS. 1 and 2 as generally having a hollow tubular body 51. However, the rigid portion 37 also could be otherwise shaped or include a series of multiple parts or sections that are substantially rigidly connected in series to form the rigid portion with different lengths as needed depending on the application of the tool. The segments 41 and the rigid portion 37 of the neck also can be made of sterilizable, durable heat-resistant injection-molded material (e.g., plastic) or may comprise other materials (e.g., metal) with anti-microbial and/or anti-bacterial additives or coatings applied thereto without departing from the invention.

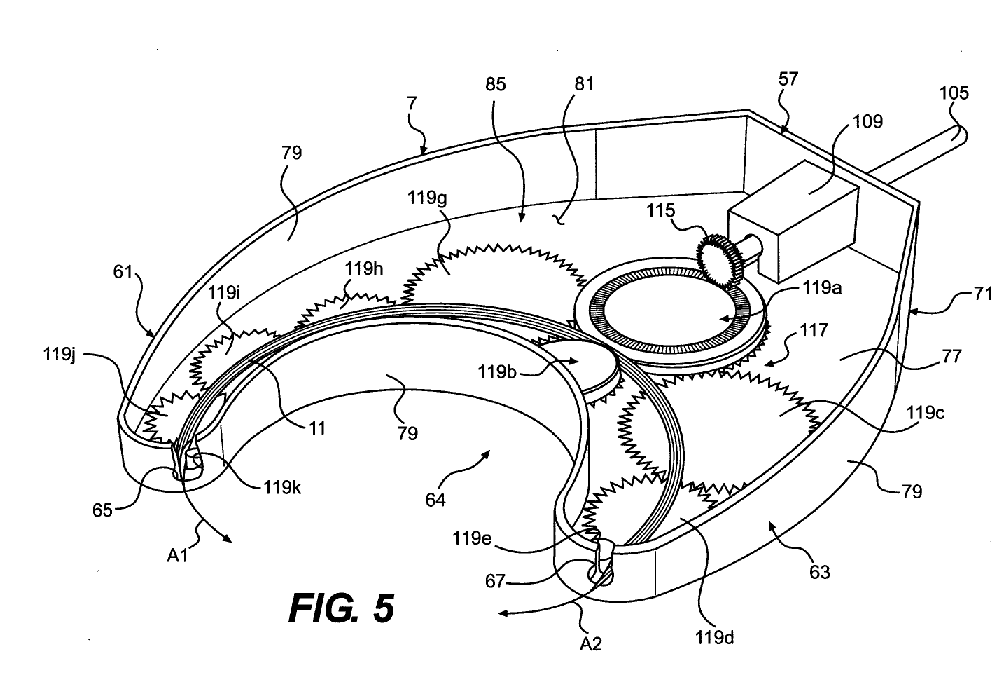

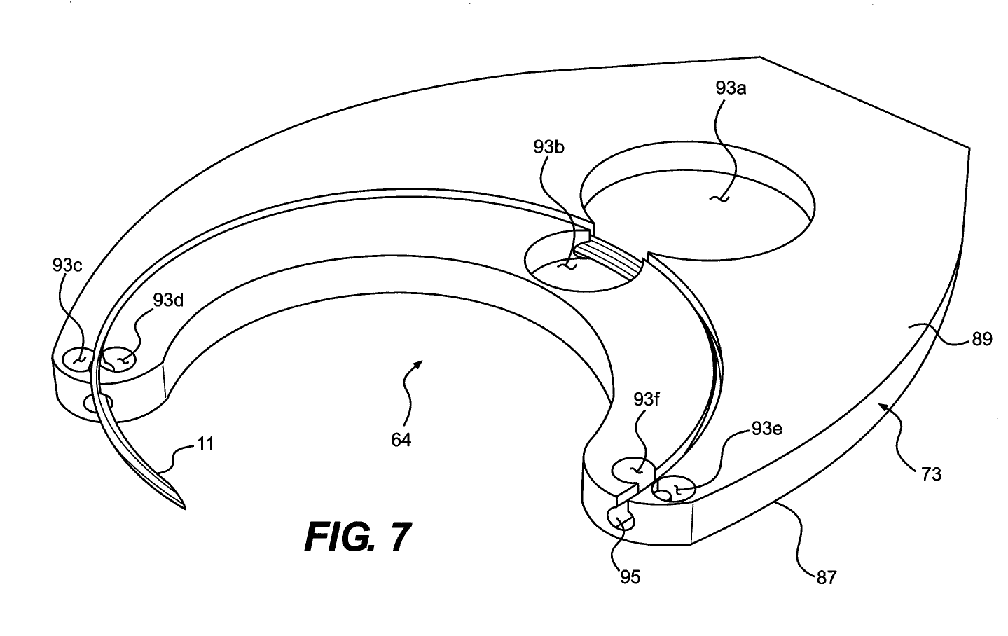

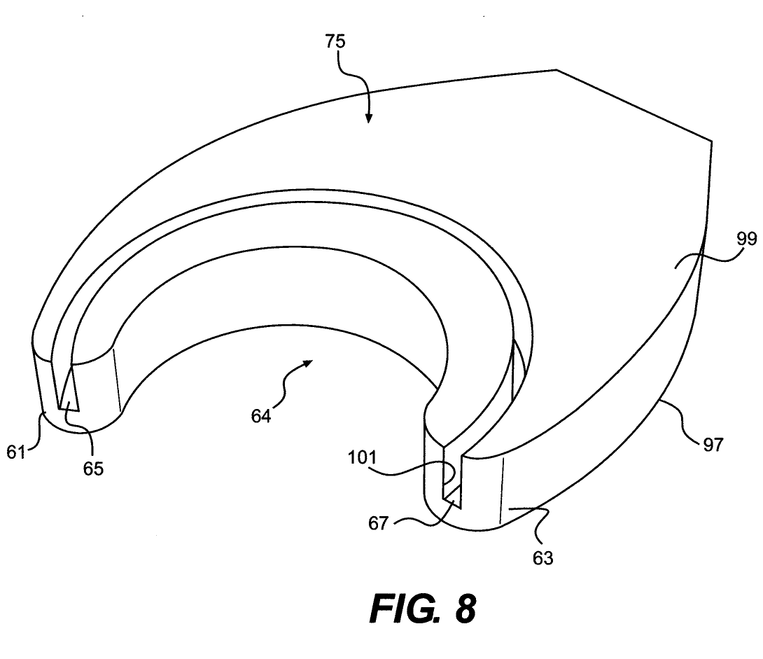

In the illustrated embodiment shown in FIGS. 1, 5 and 7-9, the head 7 is shown having a rear wall 57 connected to the flexible neck 5 and a generally C-shaped forward portion 59 having a first curved finger 61 and a second curved finger 63 defining a recess 64 within the head. Each of the curved fingers 61, 63 include a respective opening 65, 67 at a free end of the curved fingers for movement of the needle 11 (FIG. 5) relative to and within the head 7. The needle 11 can be reversibly moved through in the direction of arrows A1, A2 as indicated in FIG. 5. The head 7 further generally includes a base 71 (FIG. 5), a needle guide or groove plate 73 (FIG. 7) that is received in the base 71, and a cover 75 (FIG. 8) enclosing and forming the top surface of the head. As shown in FIG. 5, the base 71 generally has an open top and includes a bottom wall 77 and upstanding side walls 79 defining a cavity 81 or chamber within the head 7. As will be discussed in more detail below, the cavity 81 of the base 71 houses a portion of a drive mechanism 85 for moving the needle 11.

FIG. 7 shows the needle groove plate 73 in an inverted view, wherein, the needle groove plate 73 has a top surface 87 and a bottom surface 89 and includes a groove 95 defined therein that receives the needle 11 to guide and stabilize the needle as it moves about its path of travel through the head 7. The needle groove plate 73 also covers and seals the drive mechanism 85 that is located in the cavity of the head 7. The needle groove plate 73 further includes a series of substantially circular openings here indicated by 93a-f, formed in the surface 89 adjacent the groove 95 for receiving components of the needle drive mechanism 85. During operation, the needle 11 may contact tissue or other material that is transferred into the head 7 through the openings 65, 67. The needle groove plate 73 seals the cavity 81 and drive mechanism 85 therein from contact with the tissue that is received in the head 7.

The cover 75 is shown in FIG. 8 in an inverted view, wherein the cover 75 generally is sized to fit over the needle groove plate 73 to substantially fully enclose the base 71. The cover 75 has a top surface 97, a bottom surface 99, and a groove 101 formed in the bottom surface and substantially corresponding to the shape of the groove 95 of the needle groove plate 73 and the needle to allow thread or other suturing or sewing material to follow the movement of the needle 11 through the head 7. The base 71, groove plate 73, and/or cover 75 can also be otherwise shaped and configured or the head may have other components and configurations other than the embodiments illustrated and described herein.

As indicated in FIG. 5, the main operative components of the needle drive mechanism 85 are located in the head 7 for translating rotational movement of a flexible drive shaft 105 to arcuate movement of the needle 11 upon actuation of the handle controls such as trigger 27 and switch 29 (FIG. 1). As will be described later in more detail, the flexible drive shaft 105 (FIG. 3A) generally extends from the handle 3, through the neck 5, and into the head 7. In the illustrated embodiment, the flexible drive shaft 105 is held in place in the head 7 by a drive shaft block 109. The drive shaft block 109 also generally includes a bushing (not shown) to allow the drive shaft 105 to rotate and to hold the shaft in substantially fixed position.

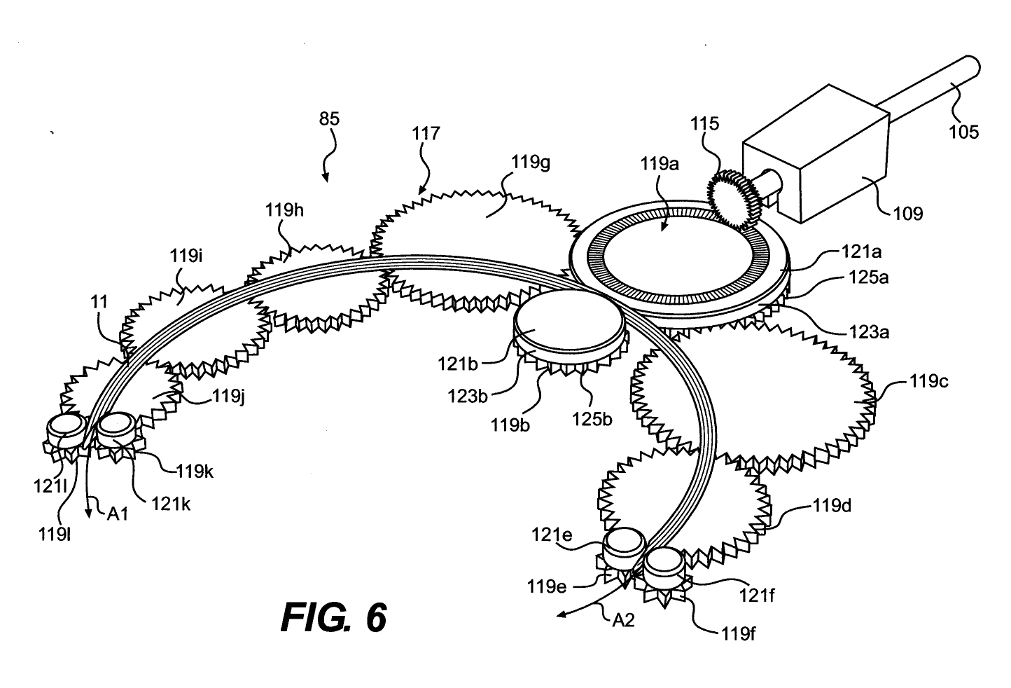

As shown in FIG. 5, at the end of the drive shaft 105 is a toothed drive gear 115 that meshes with and drives a main gear assembly 117, including a series of gears, shown in FIG. 6 as including gears 119a-119l, which are supported on the bottom wall 77 (FIG. 5) of the base 71. As FIG. 6 illustrates, the drive gear 115 attached to the drive shaft 105 generally is in a perpendicular planar alignment with the gears 119a-119l of the main gear assembly 117, with the drive gear 115 meshing with a first gear 119a of the main gear assembly 117. The first gear 119a further can have an upper portion or surface including a mating gear portion 121a that mates with the drive gear 115, and an outer lip 123a that contacts the needle 11. The outer lip 123a generally is made of a material (e.g., rubber) adapted to grip the needle 11 to urge the needle to move in the direction of rotation of the gear 119a. The lower level 125a of the first gear 119a is toothed so as to be able to mesh with corresponding gears 119e, 119g on opposite sides of the first gear and a corresponding gear 119b on the opposite side of the needle 11. The gear 119b also generally includes an upper level 121b of a similar gripping material to that of lip 123a of gear 119a and contacts the needle 11 on an opposite side from the first gear 119a. As further illustrated in FIGS. 5 and 6, the main gear assembly 117 generally is arranged in a substantially arcuate or crescent shape extending from the first gear 119a, generally in the middle of the gear assembly, to respective pairs of smaller gears 119e, 119f and 119k and 119l at the ends of the gear assembly, with the smaller gears 119e, 119f, 119k, 119l typically each having corresponding upper portions 121e, f, k and l for gripping the needle 11 and urging the needle in the direction of rotation of the smaller gears. While six contact points between the gears of gear assembly 117 and the needle 11 are generally illustrated in the illustrated embodiment as shown in FIG. 6, it will be understood that the drive mechanism also could have more or less than six contact points. Furthermore, the gear assembly 117 could be otherwise shaped, arranged and configured without departing from the invention. Still further, the needle drive mechanism 85 could include components other than gears.

In the illustrated embodiment, the needle drive mechanism 85 also includes an electric needle drive motor 131 (FIGS. 3A-3B) housed in the handle 7 for driving the flexible drive shaft 105. The trigger 27 controls operation of the needle drive motor 131 and a reversing switch also can be mounted in the handle 3 for reversing the rotation of the drive shaft 105 to actuate the needle 11 in the opposite direction A1, A2 (Figs.). As shown in FIGS. 3A-3B, the flexible drive shaft 105 extends from the needle drive motor 131 through the rigid portion 37 of the neck 5, and through the center of the segments 41 (FIGS. 2 and 4) of the flexible portion 39 of the neck, and into the head 7. As illustrated in FIG. 4, in the flexible portion 39 of the neck 5, the flexible drive shaft 105 passes through each segment 41 within a passageway 44 extending through the disk 43, the male connecting portion 47 and the female connecting portion 49. It is understood that the needle drive mechanism 85 and flexible drive shaft 105 could be otherwise arranged and include other components.

The tool 1 (FIG. 1) also includes a head positioning mechanism 141 (FIGS. 3A-3B) for moving the head to a selected position without having to reposition the handle 3. The head positioning mechanism 141 includes wires, broadly “connecting members” 145a-d (FIGS. 2 and 4), connected to the head and extending through the neck 5 and into the handle 3. In the illustrated embodiment, the wires 145a-d pass through the openings 45 in the segments 41 of the flexible portion 39 of the neck 5. The wires 145a-d generally are located inside the tubular body 51 of the rigid portion 37 of the neck 5, though they can also be covered or sealed as needed.

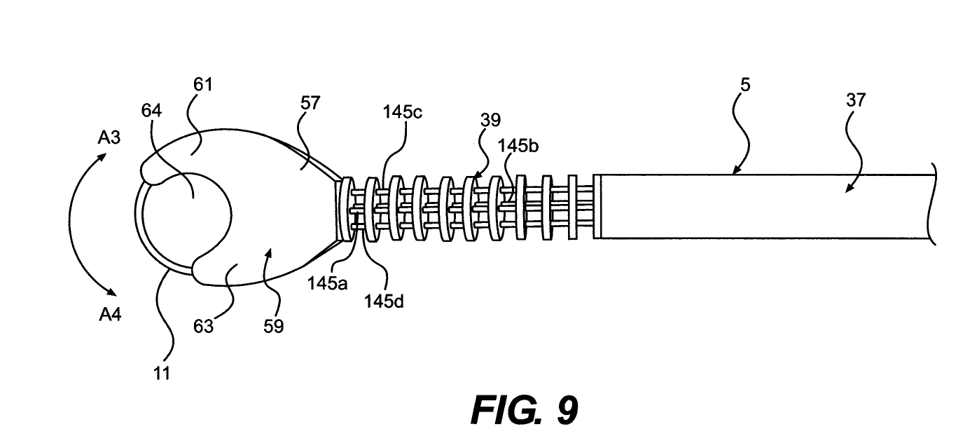

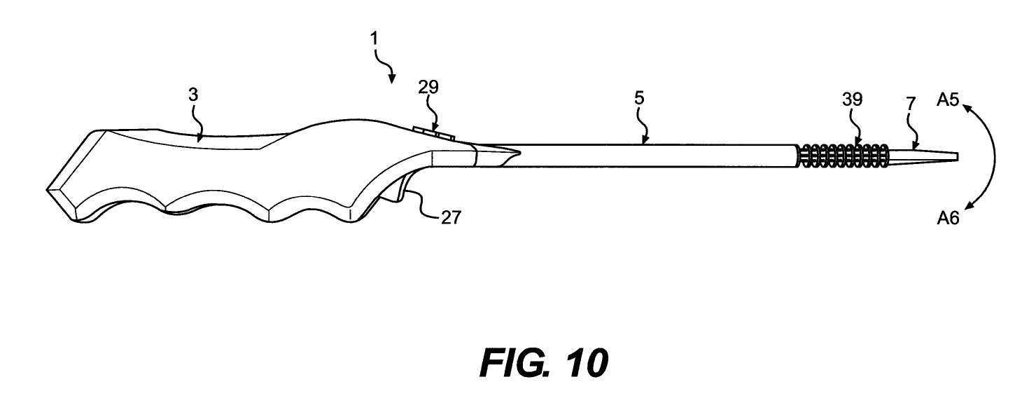

Two of the wires 145a, 145b are operable to position the head 7 in a horizontal plane by moving the head in the direction of arrows A3, A4 (as shown in FIG. 9). The other two wires 145c, 145d are operable to position the head 7 in a vertical plane by moving the head in the direction of arrows A5, A6 (FIG. 10). As shown in FIG. 3A, the head positioning mechanism 141 also can include two head positioning motors 149, 151 located in the handle 3 for selectively applying tension to the wires 145 to position the head relative to the handle. The motor 149 is connected to wires 145a, 145b for positioning the head 7 in the horizontal plane, while motor 151 is connected to wires 145c, 145d for positioning the head 7 in the vertical plane. The head positioning motors 149, 151 are actuated by operation of the four-way push button controller 29 (FIG. 1) on the top surface 15 of the handle. The controller 29 includes two buttons 155, 157 for moving the head 7 in the horizontal plane and two buttons 159, 161 for moving the head in the vertical plane. Upon actuation of a respective head positioning motor 149, 151, tension is applied to one or more of the wires 145a-d to cause the head 7 to move and the flexible portion 39 of the neck 5 to bend to allow positioning of the head relative to the handle 3. The tool 1 also could include other drives, operating mechanisms, or control devices for controlling the positioning of the head 7.

Still referring to FIG. 3A, batteries 165, 167 generally are located in the handle 3 to provide electrical power to the two motors 149, 151 of the head positioning mechanism 141 as well as the drive motor 131 for the needle drive mechanism 85. As shown in FIG. 3B, the batteries 165, 67 may be located in a different area of the handle 3 than shown in FIG. 3A, such as the area within the rear portion 21 of the handle 3. The batteries 165, 167 could be disposable so that the tool 1 could be sterilized and reused when the batteries are replaced or recharged. Alternatively, the entire tool 1 could be disposed of after use or upon loss of electrical charge of the batteries. A power source other than batteries 165, 167 also can be used.

The head positioning mechanism 141 can provide full range of motion (e.g., 360 degrees) of the head 7 in both the horizontal plane (direction of arrows A3, A4) and the vertical plane (direction of arrows A5, A6). In one alternative embodiment, the head 7 is positionable approximately 222 degrees in both the horizontal and vertical planes. However, the flexible portion 39 of the neck 5 could be lengthened or shortened by adding or subtracting segments 41 of the flexible portion of the neck to allow more or less range of motion of the head in both the horizontal and vertical planes.

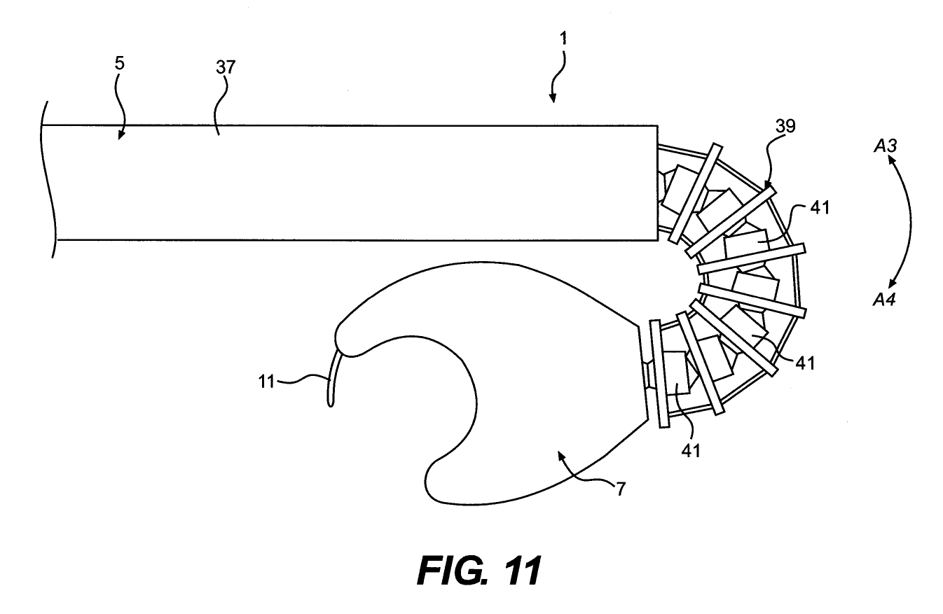

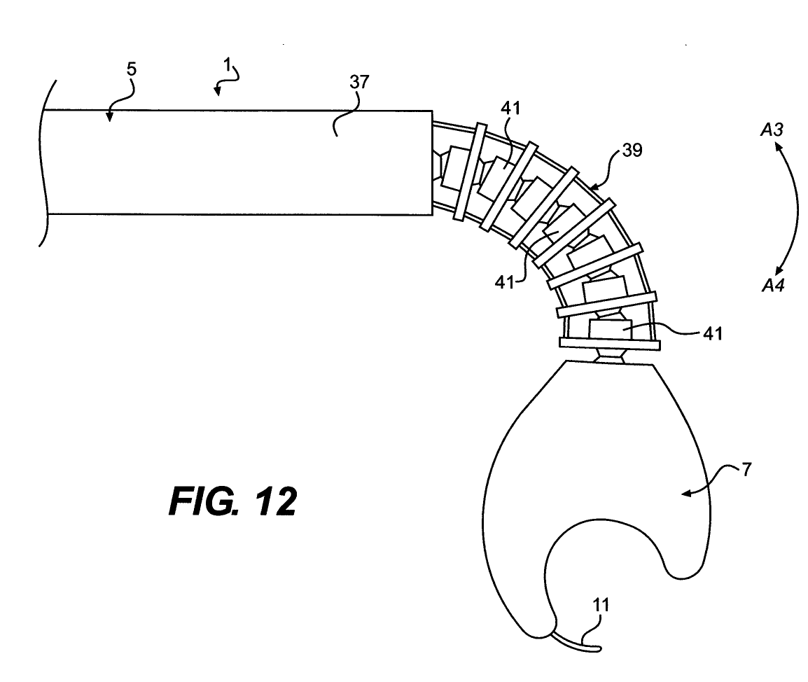

FIGS. 11-13 show various operative positions of the head in use. FIG. 11 shows the head 7 positioned at approximately 180 degrees relative to the position of the head shown in FIG. 9, with the head having been moved in the direction of arrow A4. The flexible portion 39 of the neck 5 bends to allow positioning of the head 7 from the position of FIG. 9 to the position of FIG. 11. In the illustrated embodiment, the head 7 could be repositioned approximately 360 degrees relative to the position of the head shown in FIG. 11, with the head being moved in the direction of arrow A3. As shown in FIG. 12, the head 7 is in a position that is moved approximately 90 degrees in the direction of arrow A4 from the position of FIG. 9 and is moved approximately 90 degrees in the direction of arrow A3 from the position of FIG. 11.

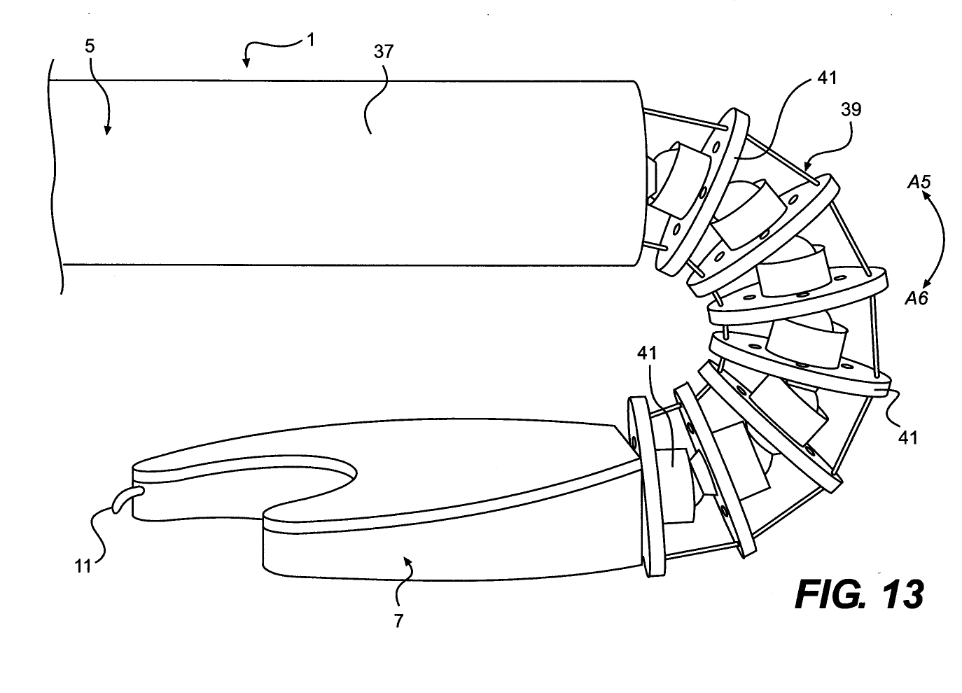

FIG. 13 shows the tool 1 with the head 7 positioned at a location displaced approximately 180 degrees relative to position of the head shown in FIG. 10, with the head having been moved in the direction of arrow A6. The head 7 further can be moved to a position approximately 360 degrees, or less, in the direction of arrow A5 from the position of the head shown in FIG. 13.

In operation, the tool 1 can be used in a variety of applications. One such application is use by a surgeon or other medical practitioner to apply sutures or tie knots. To apply sutures, a surgeon places the head 7 against the desired tissue or material for suturing. The surgeon then forms a stitch from surgical thread attached to the needle by squeezing the trigger 27 on the bottom surface 17 of the handle 3 to actuate the needle 11 in the direction of arrow Al through the tissue. The tool 1 can be used to place a single stitch or a running stitch. The head 7 can be positioned to a desired location by operating the head positioning mechanism 141 via the four-way push button controller 27 on the top surface 15 of the handle 3. Positioning the head 7 with the head positioning mechanism 141 allows the surgeon to place stitches in odd or difficult positions without having to move the handle 3 or hold the handle in an awkward position. The direction of operation of the needle 11 can be reversed by activating a reversing switch or the trigger.

To tie a knot as the surgeon comes to the end of the suturing run, the surgeon applies a first extra stitch and leaves the first extra stitch loose at the operating sight. The surgeon then makes two loops through the first extra stitch by running the needle 11 through the first extra stitch twice, and thereafter pulls the tool 1 upwards to tighten the first extra stitch. Next, the surgeon runs the needle 11 under this first extra stitch to leave a second loose extra stitch. Then, the surgeon makes two loops through the second extra stitch by running the needle 11 through the second extra stitch twice. Finally, the surgeon pulls the tool 1 upward to tighten the resulting knot. Loose ends of the suturing material can then be cut to a desired length. The pr procedure is merely one example of how to tie a knot, and it should be understood that other procedures are possible.

The tool 1 can be supplied as part of a wound closure kit such as the type to be carried by military or other personnel for emergency use at a remote location. Also, the tool 1 could be part of a robotic surgical tool that is remotely operated by a user without direct manual manipulation of the tool. Further, the tool 1 could be used in the textile, furniture and other, similar fields in various applications for mending or sewing fabrics, leathers, or other materials.

The foregoing description illustrates and describes various embodiments and features. As various changes could be made in the above construction, it is intended that all matter contained in the above description or shown in the accompanying drawings shall be interpreted as illustrative and not in a limiting sense. Furthermore, various modifications, combinations, and alterations, etc., of the above-described embodiments are contemplated by this disclosure. Additionally, while the disclosure shows and describes selected embodiments, various other non-illustrated changes, variations, combinations, modifications, and environments are within the scope of the disclosure, commensurate with the above teachings, and/or within the skill or knowledge of the relevant art. Furthermore, certain features and characteristics of each embodiment may be selectively interchanged and applied to other illustrated and non-illustrated embodiments of the invention without departing from the scope of the disclosure.

What is claimed is:

1. A surgical tool, comprising:
a handle for grasping by a user of the tool;
an elongate neck extending from the handle;
a moveable head attached to the neck;
a needle operatively connected to the head, the needle being moveable between a retracted position and an extended position;
a needle drive mechanism for moving the needle relative to the head, the needle drive mechanism comprising
a needle drive motor housed in the handle,
a drive shaft operatively connected to the needle drive motor, and
a drive assembly operatively connected to the drive shaft and the needle for converting rotation of the drive shaft into movement of the needle.

2. The tool of claim 1, wherein the drive assembly is housed in the head and comprises at least two gears configured to engage the needle for moving the needle between its retracted and extended positions.

3. The tool of claim 1, further comprising a control mechanism in the handle configured to control operation of the drive mechanism, the control mechanism comprising a switch configured to control operation of the needle drive motor.

4. The tool of claim 1, wherein the elongate neck includes a flexible portion connected to the head, the flexible portion being moveable to position the head.

5. The tool of claim 4, further comprising a head positioning mechanism configured to position the head relative to the neck.

6. The tool of claim 5 wherein the head positioning mechanism comprises:
at least two connecting members operatively connected to the head; and
at least one head positioning motor housed in the handle for controlling movements of the at least two connecting members.

7. The tool of claim 6 wherein the two connecting members are configured to pivot the head in at least one of a vertical plane and a horizontal plane.

8. The tool of claim 5, wherein the at least two connecting members comprise:
a first pair of cables configured to pivot the head relative to the vertical plane; and
a second pair of cables configured to pivot the head relative to the horizontal plane.

9. The tool of claim 4, wherein the flexible portion comprises a plurality of segments, each of said segments comprising disk, a female connecting portion disposed on a first side of the disk, and a male connecting portion disposed on a second side of the disk opposite the first side of the disk.

10. The tool of claim 9, the flexible portion further comprising:
at least two cables operatively connected to the head and extending through openings in the segments; and
at least one head positioning motor housed in the handle, the at least one head positioning motor being configured to activate the at least two connecting members to pivot the head in at least one of a vertical plane and a horizontal plane.

11. A tool for use in placing sutures and tying knots, comprising:
a handle for grasping by a user of the tool;
a neck extending from the handle, the neck comprising a flexible portion and a rigid portion;
a moveable head attached to the flexible portion;
a needle operatively connected to the head, the needle being moveable between a retracted position and an extended position;
a needle drive mechanism configured to move the needle relative to the head;
a head positioning mechanism configured to position the head relative to the neck; and
controls at least partially housed in the handle for controlling the operation of the needle drive mechanism and the head positioning mechanism.

12. The tool of claim 11 wherein the handle is shaped to facilitate grasping by a user and wherein the controls comprise:
a first control mechanism located on a top surface of the handle, the first control mechanism being operative to control the operation of the head positioning mechanism; and
a second control mechanism located along the handle, the second control mechanism being operative to control the operation of the needle drive mechanism.

13. A method of applying sutures or tying knots, comprising:
providing a tool comprising
a handle for grasping the tool,
a neck extending from the handle, the neck comprising a flexible portion and a rigid portion,
a moveable head attached to the flexible portion;
a needle operatively connected to the head;
a needle drive mechanism configured to move the needle relative to the head,
a head positioning mechanism configured to position the head relative to the neck, placing the moveable head against tissue or material;
operating the needle drive mechanism to drive the needle in a desired direction through the tissue or material to place a stitch in the tissue or material; and
operating the head positioning mechanism to adjust a position of the head in a desired location.

14. The method of claim 13, wherein operating the needle drive mechanism comprises actuating a first control mechanism to control a motor to drive the needle drive mechanism.

15. The method of claim 13, wherein operating the head positioning mechanism comprises actuating a second control mechanism to control at least one motor to position the head.

16. The method of claim 14, wherein the first control mechanism comprises a trigger located on the handle.

17. The method of claim 15, wherein the second control mechanism comprises at least one button located on the handle.
